(19) United States
(12) Patent Application Publication
(10) Pub. No.: US 2007/0225536 A1
(43) Pub. Date: Sep. 27, 2007

(54) OLEFIN CONVERSION PROCESS AND OLEFIN RECOVERY PROCESS

(76) Inventor: Eugene Frederick LUTZ, Houston, TX (US)

Correspondence Address: SHELL OIL COMPANY, P O BOX 2463, HOUSTON, TX, 772522463, US

(21) Appl. No.: 11/689,252

(22) Filed: Mar. 21, 2007

Related U.S. Application Data
(60) Provisional application No. 60/785,340, filed on Mar. 23, 2006.

(51) Int. Cl. C07C 6/00

(52) U.S. Cl. 585/643

(57) ABSTRACT

The present invention provides a process for converting olefins from a mixture of olefins and non-olefinic organic compounds of comparable boiling point to olefin products with a larger difference in boiling point from the boiling point of the non-olefinic organic compounds. Additional steps may be performed to recover the olefin product including separating the olefin product from the mixture produced in the conversion step.

This application claims priority to U.S. provisional application Ser. No. 60/785,340, filed Mar. 23, 2006, the entire disclosure of which is hereby incorporated by reference.

### FIELD OF THE INVENTION

This invention relates to a method for converting olefins and for recovering olefins.

### BACKGROUND OF THE INVENTION

It is known that it is somewhat difficult to separate olefins from paraffins of the same carbon number. In commercial practice, one process by which olefins have been produced involves recovering them from mixtures of olefins and paraffins. UOP's Pacol process of paraffin dehydrogenation is used to produce olefins but the conversion is rather low (10 to 15 percent) and the paraffin must be recovered for recycle. In the Pacol process, an n-paraffin of the desired chain length is dehydrogenated in a catalytic fixed-bed reactor at low pressure and moderately high temperature.

Certain commonly used current commercial processes separate the olefin by combining the Pacol process with the capital intensive UOP Olex adsorption process to obtain olefins, including higher olefins. The Olex process provides large scale bulk separation from the liquid phase of olefins from paraffins by countercurrent flow of the liquid and the adsorbent without actual movement of the adsorbent bed. The combined process is generally referred to as the Pacol-Olex process which is described in Hydrocarbon Process, 58 (11), 185 (1979). This technology is practiced in linear alkyl benzene manufacture.

Another commercial olefin recovery process involves converting the olefin to a higher boiling derivative which can be separated by distillation. This technology is also practiced in linear alkyl benzene manufacture.

It can be seen that it would be advantageous to provide a separation process which is not capital intensive and may leave the olefin underivatized, especially because an olefin is a desired product.

### SUMMARY OF THE INVENTION

The present invention provides a process for the conversion of olefins in admixture with non-olefinic organic compounds and also relates to a process for recovering the converted olefins. In one embodiment, the olefins may be converted into other olefins to increase the difference in boiling point of the olefins from the boiling point of the non-olefinic organic compounds. The converted olefins may then be more easily separated from the non-olefinic organic compounds than the original olefins. The invention also provides a process for separating olefins in a mixture of olefins and non-olefinic organic compounds for which the difference in the boiling points is small enough, such as about 10° C. or less, to make it difficult to separate them by means such as distillation.

In another embodiment, the process of this invention comprises:
(a) providing a mixture comprising feed olefins and non-olefinic organic compounds, and
(b) converting the feed olefins in the mixture to converted olefins for which the difference in boiling point from the boiling point of the non-olefinic organic compounds is larger than the difference in boiling point of the feed olefins from the boiling point of the non-olefinic organic compounds.

The converted olefins may be higher or lower boiling olefins products, i.e., they may have higher or lower boiling points than the feed olefins. A mixture of higher and lower boiling olefin products may be also produced.

Another embodiment of the present invention relates to the conversion of olefins in a mixture of olefins and paraffins by metathesis and also relates to a process for recovering the olefins. The olefins and paraffins may have a difference in the boiling points which is small enough, for example, about 10° C. or less, which makes it difficult to separate them by means such as distillation, such as, for example, when the olefins and paraffins are of the same carbon number. The olefins may be converted into other olefins that may then be more easily separated from the paraffins.

In another embodiment, the process comprises:
(a) providing a mixture comprising feed olefins and paraffins, and
(b) metathesizing the olefins in the mixture to converted olefins to increase the difference in boiling point of the olefins from the paraffins, preferably thereby producing lower boiling olefin products and higher boiling olefin products for which the difference in boiling point from the boiling point of the paraffins is larger than the difference in boiling point of the feed olefins from the boiling point of the paraffins.

In another embodiment of this invention, in either of the processes described immediately above an additional process step c) of recovering the olefin products may be performed. Step c) may comprise separating the converted olefin products from the mixture produced in step b) by distillation, flashing or other conventional means. In another embodiment of this invention, at least a portion of the converted olefins may be removed from the mixture produced in step b) as they are formed. In all embodiments, lower boiling olefin products may be removed from the mixture produced in step b) as they are formed. Higher boiling olefin products may then be removed. In another embodiment where a mixture of lower and higher boiling olefin products is produced in step b), the lower boiling olefin products may be separated from the mixture produced in step b) in step c) (i) and the higher boiling olefin products may be separated from the remaining material in step c) (ii).

In another embodiment, at least one of the olefin products may be hydroformylated to produce alcohols. In another embodiment, the alcohols may be alkoxylated to produce alcohol alkoxylates. In another embodiment, the alcohols and/or the alkoxylates may be sulfated to produce alcohol sulfates and/or alcohol alkoxysulfates. In another embodiment, at least one of the olefin products may be reacted with aromatic hydrocarbons to produce alkyl aromatic hydrocarbons which may be sulfonated to produce alkylarylsulfonates. In another embodiment, the alcohol sulfates and/or alcohol alkoxysulfates and/or alkylarylsulfonates may be combined with conventional detergent additives to produce detergent compositions In another embodiment, at least one of the olefin products is sulfated to produce and olefin sulfate.

### DETAILED DESCRIPTION OF THE INVENTION

The olefins in the starting mixture with the organic non-olefinic compounds (for example, paraffins) may be converted to olefins having different boiling points by several means. The olefins may be subjected to metathesis. They may be subjected to skeletal isomerization or dimerization conditions.

The metathesis reaction has also been referred to as olefin disproportionation, double decomposition, and double displacement. In the present process, the reaction may be self-metathesis of the olefins to form a lower boiling olefin product and a higher boiling olefin product. Internal olefins are preferred for use herein because their products are more useful than the products of the metathesis of alpha olefins. The products of mid-chain internal olefins are the most useful. In the present invention, the more volatile olefins produced comprise the lower boiling olefin products which may be removed as they are formed. The lower boiling olefin products have boiling points lower than that of the feed olefins and paraffins in the mixture. The less volatile olefins produced comprise the higher boiling olefin products and these products have boiling points higher than the boiling points of the feed olefins and paraffins. The higher boiling olefin product may be a mid-chain olefin which is an olefin wherein the double bond is at or near the middle of the chain, for example no more that 3 carbons from the middle of the chain, preferably no more than 2 carbons from the middle of the chain. The location of the double bond in the chain can be determined by nuclear magnetic resonance spectrometry (NMR) or mass spectrometry.

In order to obtain faster and more complete reaction and then separation of the olefins from the non-olefinic organic compounds, it may be desirable to create non-equilibrium conditions in the conversion reaction, particularly in the metathesis reaction. Non-equilibrium conditions may be created in this process simply by separating the converted low boiling olefins from the mixture of step b) as it is formed. Non-equilibrium conditions are preferred to enable the maximum production of one set of metathesis products, a preferred result herein. For example, a C₉ internal olefin may self-metathesize to produce a C₁₄ internal olefin and a C₄ olefin—a set of metathesis products whose production is maximized under non-equilibrium conditions. If the reaction is at equilibrium, the metathesis products will continue to react to produce other metathesis products until the equilibrium point is reached. Many metathesis products will be present in the mixture and some of them will be olefins of such low or high carbon numbers that they are not preferred products of this process. Preferably, olefin products of C₄₋₂₀ are produced according to the process of the invention, more preferably C₆₋₁₆.

A wide variety of olefins may be used in the starting or feed mixture in the process of the present invention. In order to produce products which are currently more valuable, olefins containing from about 6 to about 30 carbon atoms, preferably from about 6 to about 24 carbon atoms, may be used in the present invention. In a particularly preferred embodiment, the olefins may contain from about 6 to about 22 carbon atoms. These olefins may be linear or branched, but are preferably linear or lightly branched, such as olefins comprising methyl branching, preferably no more than 20 mol % methyl branching, because the olefins' double bonds are usually at the site of the branch and such bonds are more stable and thus the metathesis reaction proceeds more slowly.

The olefin and the non-olefinic organic compounds in the mixture of a) may have boiling points that are sufficiently close that it is difficult to separate them by means such as distillation or flashing. The olefins and non-olefinic organic compounds, such as paraffins, may be difficult to separate if the boiling point difference is about 10° C. or less. Costly distillation equipment may be required and purity problems may occur if the difference is about 5° C. or less. Distillation may not be used if the difference is about 1° C. or less. This is generally true for any mixture of olefins and any non-olefinic organic compounds. In the process of the present invention, the olefins are preferably converted to other olefins which have a boiling point difference from the non-olefinic organic compounds which is sufficient to allow them to be separated by means such as distillation or flashing. If the difference is more than about 10° C., distillation can be used to separate the converted olefins and the non-olefinic organic compounds. Subject to the need for costly distillation equipment and the possibility of purity problems, the difference may be more than about 5° C.

Paraffins are used in one embodiment of the invention wherein the mixture provided in step a) is a mixture of olefins and paraffins The olefins and paraffins may be of the same carbon number.

Olefins and paraffins of the same carbon number in step a) are difficult to separate because their boiling points are very close and it is too difficult to separate them by conventional distillation and/or flashing methods. According to the process of this invention, the olefins may be converted into a mixture of lower boiling olefin products and higher boiling olefin products. The lower boiling olefin products have boiling points lower than the boiling points of the feed olefins and paraffins, preferably sufficiently low enough that the recovery of the lower boiling olefin products may be carried out by conventional distillation and/or flashing methods. The higher boiling olefin products have boiling points higher than the boiling points of the feed olefins and paraffins, preferably sufficiently high enough that the recovery of the higher boiling olefin products may be carried out by conventional distillation and/or flashing methods.

Single carbon number cut (comprising primarily, i.e., more than about 80% by weight or more than about 90% by weight, olefins and paraffins which have one carbon number) mixtures of olefins and paraffins are preferred for use in the process of this invention because most of the olefin products will have different a carbon number than the feed olefins. A 2 carbon number cut may also be used advantageously even though some part of the product olefins may have the same carbon number as the feed olefins and paraffins and thus may have to be recycled. A three carbon cut may require that about 15% by weight or more of the product olefins be recycled and a 4 or more carbon number cut may require that an even higher amount of the product olefins be recycled.

For practical reasons of availability, it may be preferable to use in the present process internal olefins in the C₆ to C₂₄ range. Internal olefins are typically produced commercially by chlorination-dehydrochlorination of paraffins, by paraffin dehydrogenation (such as the Pacol process), and by isomerization of alpha olefins. The resulting internal olefin products are generally substantially of linear nature. Linear internal olefin products in the C₈ to C₂₄ range are marketed by Shell Chemical LP. These commercial products typically contain about 70 percent by weight or more, most often about 80 percent by weight or more, linear mono-olefins in a specified carbon number range (e.g., C₁₀ to C₁₂, C₁₁ to C₁₅, C₁₂ to C₁₃, C₁₅ to C₁₈, etc.), wherein the remainder of the product usually comprises olefins of other carbon numbers or carbon structure, diols, paraffins, aromatics, and other impurities resulting from the synthesis process. Internal olefins in the C₆ to C₂₂ range may be considered most preferred for use in the olefin/paraffin feed, primarily because of the useful products that can be made from such internal olefins. The products which can be made from the internal olefins separated by the process of the present invention include surfactants, detergents, oilfield chemicals, synthetic motor oils, and others.

In general, any disproportionation or metathesis catalyst may be utilized in step (b) of the present process. There are a wide variety of catalysts which have been employed for conducting disproportionation reactions including any known olefin metathesis catalyst, examples of which are described in WO 01/05735 and U.S. Pat. No. 7,041,864, which are incorporated herein by reference in their entirety. Useful catalysts suitably comprise one or more metals selected from the group consisting of Cr, Mo, W, Mn, Tc, Re, Fe, Ru, Os. Preferably, the catalyst comprises one or more metals selected from the group consisting of Mo, W, Re, and Ru, more preferably a metal selected from the group consisting of Re and Ru. Most preferably, the catalyst comprises Re.

Another list of useful catalysts is included in my U.S. Pat. No. 5,672,802, which is incorporated herein by reference in its entirety. The catalysts described therein include inorganic refractory materials containing molybdenum and/or tungsten oxide. Such catalysts may also contain a promoter to enhance the disproportionation catalyst activity. Elemental metal promoters selected from the group consisting of barium, magnesium, tungsten, silver, antimony, zinc, manganese, and tin may be used. In addition, organometallic compounds, such as aluminum and tin alkyls may be used to promote solid catalysts including molybdenum and rhenium oxide. Suitable support materials include, but are not necessarily limited to, alumina, silica, molecular sieves, such as zeolites, activated carbon, aluminosilicate clays, amorphous silicoaluminas, and the like. Preferred supports are aluminum oxide (for Mo and Re) and silicon oxide (for W). In fact, any new metathesis catalyst developed in the future should also be useful in the present invention.

Suitable catalysts include homogeneous and heterogeneous catalyst systems. Suitable homogeneous catalysts include, but are not necessarily limited to “Grubbs” catalysts, Schrock catalysts, and a variety of tungsten based catalysts Schrock catalysts (based on Mo) are commercially available from Strem Chemicals. Suitable tungsten-based metathesis catalyst precursors and activators also are available from Strem Chemicals. An example of a tungsten-based metathesis catalyst precursor is a tungsten halide, such as tungsten hexachloride. Suitable activators include, but are not necessarily limited to alkyl metals, such as the promoters listed below. Preferred activators are alkyl aluminums, preferably trialkyl aluminums. Due primarily to ease of separation of the final product, preferred catalysts are heterogeneous.

In one embodiment of this invention the metathesis catalyst may be a non-isomerizing catalyst. A non-isomerizing catalyst is preferred because isomerization likely will move enough of the double bonds of the initial olefins before metathesis that the result may be the production of the olefins discussed above that are of such low or high carbon numbers that they are not preferred products of this process.

In one embodiment, the non-isomerizing catalysts for use in the metathesis step of the present invention are heterogeneous catalysts in which rhenium, molybdenum and/or tungsten is deposited on a support of silica, alumina, or alumina phosphate. One such heterogeneous catalyst is rhenium deposited on alumina, especially Re/Al₂O₃ comprising Re at a concentration of from about 1 to about 20% wt %, preferably from about 5 to about 12 wt. %, more preferably at about 10 wt %. Heterogeneous molybdenum catalysts may include an alkali metal to minimize the amount of isomerization. Tungsten on silica catalysts, such as those described in U.S. Pat. Nos. 6,683,019 and 6,727,396, which are herein incorporated by reference in their entirety, may also be used in the process of the invention.

Non-isomerizing homogeneous catalysts based on ruthenium may also be used in the process of the invention. Such catalysts include Grubbs catalyst. The following are examples of suitable Grubbs catalysts:

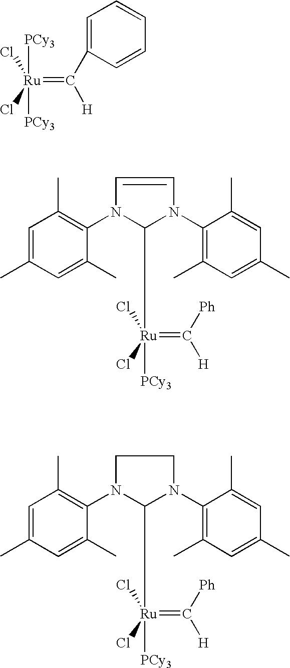

### The foregoing Grubbs catalysts are commercially available, for example, from Strem Chemicals and Aldrich Chemicals.

Prior to its use, the catalyst is typically activated by calcination carried out in a conventional manner. Particularly suitable catalysts for use in this invention include molybdenum oxide and/or rhenium oxide supported on alumina.

Metathesis conditions which may be used include those which are described in U.S. Pat. No. 5,672,802, WO 01/105735, and U.S. Pat. No. 7,041,864, which are herein incorporated by reference in their entirety. The reaction conditions, including temperature, pressure, flow rates, etc., will vary somewhat depending upon the specific catalyst composition used, the particular feed mixture which is used, etc. Generally, metathesis may be carried out at temperatures ranging from about −10° C. to about 300° C. Pressures in the range of about 0.01 kPa to about 300 kPa may be used. If the temperature is above about 300° C., too much isomerization of the olefins may occur. Metathesis is usually carried out in a liquid phase and liquid reaction diluents may be used. Examples of suitable diluents are saturated hydrocarbons. If the diluent is used, it is usually used in amounts up to 20 moles of diluent per mole of olefin/paraffin mixture.

If a tungsten-based catalyst is used, the temperature may range from about 200 to about 300° C. If a molybdenum-based catalyst is used, the temperature may range from about 100 to about 150° C. If a rhenium-based catalyst is used, the temperature may range from about 30 to about 60° C.

The olefins in the mixture of olefins and paraffins, preferably of the same carbon number, may be metathesized to produce lower boiling olefin products and higher boiling olefin products. The lower and higher boiling olefin products may be removed from the reaction in step c), which may comprise steps c) (i) and c) (ii).

Separation step (c) (i) may utilize distillation, flashing or a similar method to remove the lower boiling olefin products. At the end of steps b) and c) (i), what remains may be a mixture of paraffins and higher boiling olefin products, as well as any impurities that may have been in the feed mixture. The higher boiling olefin products may then be separated from the paraffins in step c) (ii) which may utilize distillation, flashing or a similar method to remove the higher boiling olefin products from the paraffins.

Alternatively, the lower boiling olefin products may be removed as they are formed in step b), thereby creating non-equilibrium conditions which may assist in driving the metathesis reaction. Step c) may then comprise the separation of the higher boiling olefin products from the paraffins.

The paraffins may then be purified and recycled or used for another purpose. The lower and higher boiling olefin products may then be used for a variety of applications and in a variety of further reactions to produce other products One example is reaction of the olefin with alkylene in the presence of a catalyst to form an alpha olefin product.

All of the steps of this process may advantageously be carried out in the same reaction vessel. Steps a) and b) may be carried out in one reaction vessel and step c) in a second reaction vessel If higher and lower boiling olefin products are produced, step c) (i) may take place in a second reaction vessel, and step c) (ii) in the second reaction vessel or in a third reaction vessel. In order to achieve the maximum advantage from non-equilibrium conditions, it is preferred that steps b) and c) (i) be carried out together at the same time, preferably in the same reaction vessel.

Skeletal isomerization may be used as the method for converting the starting olefins to other olefins which have a greater difference in boiling point from the non-olefinic organic compounds. As those skilled in the art will appreciate, the expression “skeletal isomerization”, as used herein, refers to a rearrangement of the carbon structure of an olefinic hydrocarbon and is to be distinguished from double bond or geometric isomerization, which involves a shift of a hydrogen atom from one carbon to another in an olefin chain.

The isomerization conditions used herein may be chosen from a wide variety of catalysts and isomerization processes. Some of these processes include those described in U.S. Pat. Nos. 3,786,112, 4,749,819, 4,727,203, 5,107,047, 5,177,281, and 5,510,306, the disclosures of which are all herein incorporated by reference in their entirety. The conditions may include operating at a temperature of from about 0 to about 500° C., a pressure from about 1 to about 10,000 kPa, and, in a continuous process, a weight hourly space velocity of from about 0.1 to about 100. Generally, temperatures of about 200° C. or less may be sufficient and pressures of from about atmospheric to about 5000 kPa may be used.

Almost any isomerization catalyst may be used. Among the isomerization catalysts that may be used are the catalysts which are disclosed in U.S. Pat. Nos. 3,786,112, 4,749,819, 4,727,203, 5,107,047 5,177,281, and 5,510,306, which are incorporated by reference.

Suitable isomerization catalysts for use in this invention include catalysts comprising Group VIII noble metals, i.e., palladium, platinum, or ruthenium; niobium, or vanadium oxides; Group I, Group II, or Group III metal oxides including sodium oxide, potassium oxide, magnesium oxide, calcium oxide, zinc oxide, gamma-alumina, bauxite, eta-alumina, barium oxide, strontium oxide and mixtures thereof; and Group I metal carbonates on alumina.

Other isomerization catalysts which may be used include alumino silicate catalysts. A preferred alumino silicate catalyst is a ferrierite alumino silicate catalyst defined as having eight and ten member ring channels. Other preferred alumino silicates are ferrierite catalysts which are exemplified by the ZSM-35 alumino silicate described in U.S. Pat. No. 4,016,245, the disclosure of which is incorporated herein by reference in its entirety, or by a piperidine derived ferrierite as described in U.S. Pat. No. 4,251,499, the disclosure of which is herein incorporated by reference in its entirety. Other useful zeolites include Theta-1, ZSM-12, ZSM-22, ZSM-23, and ZSM-48. These alumino silicates may be associated with a catalytic metal, preferably selected from Group VIII or Group VIB of the periodic table. These metals may be exemplified by palladium, platinum, ruthenium, nickel, cobalt, molybdenum, osmium, and may be present in combination with one another. These catalytic metals may be present in quantities from about 0.1 weight percent to about 25 weight percent of the total catalyst composition.

The ZSM-22 catalyst is more particularly described in U.S. Pat. No. 4,556,477, the entire contents of which are herein incorporated by reference. The ZSM-23 catalyst is more particularly described in U.S. Pat. No. 4,076,842, the entire contents of which are herein incorporated by reference. The MCM-22 catalyst described in U.S. Pat. No. 5,107,047 may also be used as the isomerization catalyst in the present invention. All of these patents are herein incorporated by reference.

Dimerization is another method which may be used as the method for converting the starting olefins to other olefins which have a greater difference in boiling point from the non-olefinic organic compounds. The dimerization reaction may be operated at temperatures up to about 200° C., preferably from about −10 to about 100° C., and more preferably from about 10 to 50° C. The pressure may range from about 1 to about 10,000 kPa, preferably from atmospheric pressure to about 5000 kPa.

There are a variety of dimerization catalysts which may be used in the present invention. These catalysts include those described in U.S. Pat. Nos. 4,252,987, 4,859,646, 6,222,077, 6,291,733, and 6,518,473, all of which are herein incorporated by reference. One such catalyst may comprise a dicyclopentadienyl halogenated titanium compound, an alkyl aluminum halide, and a nitrogen Lewis phase. Other such catalysts may include 1) a palladium compound, 2) a chelate ligand comprising a compound containing at least 2 nitrogen atoms which are connected through a chain comprising two or more carbon atoms, 3) a protonic acid, and 4) a salt of copper, iron, zinc, tin, manganese, vanadium, aluminum, or a group VIB metal. In another embodiment, the catalyst may be one wherein a metal, preferably nickel, is bound to at least one hydrocarbyl group or a catalyst which consists of complexes formed by admixing at least one nickel compound with at least one alkyl aluminum compound and optionally a ligand. The catalyst may also be a catalyst comprising a combination of a nickel carboxylate or a nickel chelate with an alkyl aluminum halide or an alkyl aluminum alkoxide. Furthermore, catalysts for dimerization may be virtually any acidic material including zeolites, clays, resins, BF₃ complexes, HF, H₂SO₄, AlCl₃, ionic liquids, super acids, etc.; and preferably a group VIII metal on an inorganic oxide support such as a zeolite support. Another dimerization catalyst which may be used in the present invention is the transition metal catalyst/activating cocatalyst described in U.S. Pat. No. 6,291,733, which is herein incorporated by reference in its entirety.

Many olefin derivatives may be made from the converted olefins of this invention.

Alcohols derived from long chain olefins have considerable commercial importance in a variety of applications, including detergents, soaps, surfactants, freeze point depressants and lubricating oils, emollients, agricultural chemicals, and pharmaceutical chemicals. These alcohols are produced by any one of a number of commercial processes including the Oxo process and the hydroformylation of long chain olefins.

The olefins of this process may be converted into alcohols by the process described in U.S. Pat. No. 4,472,525 (nickel catalysts), U.S. Pat. Nos. 5,849,960, and 6,710,006 (Fe bisiminepyridine complex catalysts), all of which are herein incorporated by reference in their entirety. Olefins may be converted into alcohols by hydroformylation, preferably with synthesis gas (CO+H₂), in the presence of a hydroformylation catalyst. In addition to the processes and catalysts described in the patents above, many other well-known hydroformylation processes and catalysts may also be used to convert the olefins of the present invention into alcohols.

The alcohols may be suitable for the manufacture of anionic, nonionic, and cationic surfactants. Specifically, the alcohols can be used as the precursor for the manufacture of anionic sulfates, including alcohol sulfates and oxylakylated alcohol sulfates, and nonionic oxyalkylated alcohols.

Any technique known for sulfating alcohols may be used herein The alcohols may be directly sulfated or first oxyalkylated followed by sulfonation. The olefin products of this invention may also be directly sulfated.

The general class of anionic surfactants includes alcohol sulfates which may be characterized by the chemical formula:

R′—O—SO₃M⁺

and includes alcohol alkoxysulfates which may be characterized by the chemical formula:

R′—O—(R—O)ₓ—SO₃M³⁰

wherein R′ represents the olefin moiety, R is an alkyl group, such as ethyl, propyl, butyl, and the like, x represents the average number of oxyalkylene groups per molecule and is in the range of from about 0 to about 12, and M is a cation selected from an alkali metal ion, an ammonium ion, and mixtures thereof. Of course, the surfactant may by oxyalkylated with any oxirane containing compound other than, in mixture with, or sequentially with ethylene oxide, propylene oxide and the like.

Sulfonation processes are described, for instance, in U.S. Pat. No. 3,462,525, issued Aug. 19, 1969 to Levinsky et. al., U.S. Pat. No. 3,428,654 issued Feb. 18, 1969 to Rubinfeld et. al., U.S. Pat. No. 3,420,875 issued Jan. 7, 1969 to DiSalvo et. al., U.S. Pat. No. 3,506,580 issued Apr. 14, 1970 to Rubinfeld et. al., U.S. Pat. No. 3,579,537 issued May 18, 1971 to Rubinfeld et, al., and U.S. Pat. No. 3,524,864 issued Aug. 18, 1970 to Rubinfeld, each incorporated herein by reference. Suitable sulfonation procedures include sulfur trioxide (SO₃) sulfonation, chlorosulfonic acid (ClSO₃H) sulfonation and sulfamic acid (NH₂SO₃H) sulfonation. When concentrated sulfuric acid is used to sulfate alcohols, the concentrated sulfuric acid may be typically from about 75 percent by weight to about 100 percent by weight, preferably from about 85 percent by weight to about 98 percent by weight, in water. Suitable amounts of sulfuric acid may be generally in the range of from about 0.3 mole to about 1.3 moles of sulfuric acid per mole alcohol, preferably from about 0.4 mole to about 1.0 mole of sulfuric acid per mole of alcohol.

A typical sulfur trioxide sulfonation procedure may include contacting liquid alcohol or its ethoxylate and gaseous sulfur trioxide at about atmospheric pressure in the reaction zone of a falling film sulfator cooled by water at a temperature in the range of from about 25° C. to about 70° C. to yield the sulfuric acid ester of alcohol or its ethoxylate. The sulfuric acid ester of the alcohol or its ethoxylate then may exit the falling film column and may be neutralized with an alkali metal solution, e.g., sodium or potassium hydroxide, to form the alcohol sulfate salt or the alcohol ethoxysulfate salt.

Suitable oxyalkylated alcohols may be prepared by adding to the alcohol or mixture of alcohols to be oxyalkylated a calculated amount, e.g., from about 0.1 percent by weight to about 0.6 percent by weight, preferably from about 0.1 percent by weight to about 0.4 percent by weight, based on total alcohol, of a strong base, typically an alkali metal or alkaline earth metal hydroxide such as sodium hydroxide or potassium hydroxide, which serves as a catalyst for oxyalkylation. Other catalysts, including lithium hydroxide, magnesium hydroxide, magnesium oxide, calcium oxide, and alumina oxide, may also be used. The resulting mixture may be dried, such as by vapor phase removal of any water present, and an amount of alkylene oxide calculated to provide from about 1 mole to about 12 moles of alkylene oxide per mole of alcohol may be then introduced and the resulting mixture may be allowed to react until the alkylene oxide is consumed, the course of the reaction being followed by the decrease in reaction pressure.

The oxyalkylation may be conducted at elevated temperatures and pressures. Suitable reaction temperatures may range from about 120° C. to about 220° C. with the range of from about 140° C. to about 160° C. being preferred. A suitable reaction pressure may be achieved by introducing to the reaction vessel the required amount of alkylene oxide which has a high vapor pressure at the desired reaction temperature. For consideration of process safety, the partial pressure of the alkylene oxide reactant may preferably be limited, for instance, to less than about 60 psia, and/or the reactant is preferably diluted with an inert gas such as nitrogen, for instance, to a vapor phase concentration of about 50 percent or less. The reaction may, however, be safely accomplished at greater alkylene oxide concentration, greater total pressure and greater partial pressure of alkylene oxide if suitable precautions, known to the art, are taken to manage the risks of explosion. With respect to ethylene oxide, a total pressure of between about 0.25 and 0.75 MPa, with an ethylene oxide partial pressure between about 0.1 and 0.5 MPa, may be used, while a total pressure of between about 0.3 and 0.6 MPa, with an ethylene oxide partial pressure between about 0.15 and 0.35 MPa, may also be used. The pressure serves as a measure of the degree of the reaction and the reaction is considered to be substantially complete when the pressure no longer decreases with time.

It should be understood that the oxyalkylation procedure may serve to introduce a desired average number of alkylene oxide units per mole of alcohol oxyalkylate. For example, treatment of an alcohol mixture with 3 moles of ethylene oxide per mole of alcohol may serve to effect the ethoxylation of each alcohol molecule with an average of 3 ethylene oxide moieties per mole alcohol moiety, although a substantial proportion of alcohol moieties may become combined with more than 3 ethylene oxide moieties and an approximately equal proportion may have become combined with less than 3. In a typical ethoxylation product mixture, there may also be a minor proportion of unreacted alcohol.

Other alkyene oxides may be used, such a propylene oxide and butylene oxide. These may be added as a mixture to the alcohol or sequentially to make a block structure.

The sulfated alcohol compositions made this way may be used as surfactants in a wide variety of applications, including detergents such as granular laundry detergents, liquid laundry detergents, liquid dishwashing detergents; and in miscellaneous formulations such as general purpose cleaning agents, liquid soaps, shampoos and liquid scouring agents.

The sulfated alcohol and alkoxyalcohol compositions may be particularly useful in detergents, specifically laundry detergents. These are generally comprised of a number of components besides the sulfated alcohol composition of the invention:

other surfactants of the ionic, nonionic, amphoteric or cationic type, builders (phosphates, zeolites), cobuilders (polycarboxylates), bleaching agents and their activators,

foam controlling agents, enzymes, anti-greying agents, optical brighteners, and stabilizers.

Liquid laundry detergents generally comprise the same components as granular laundry detergents, but generally contain less of the inorganic builder component. Hydrotropes are often present in the liquid detergent formulations. General purpose cleaning agents may comprise other surfactants, builders, foam suppressing agents, hydrotropes and solubilizer alcohols.

In addition to surfactants, washing and cleaning agents may contain a large amount of builder salts in amounts up to 90% by weight, preferably between about 5 and 35% by weight, to intensify the cleaning action. Examples of common inorganic builders are phosphates, polyphosphates, alkali metal carbonates, silicates and sulfates. Examples of organic builders are polycarboxylates, aminocarboxylates such as ethylenediaminotetraacetates, nitrilotriacetates, hydroxycarboxylates, citrates, succinates and substituted and unsubstituted alkanedi- and polycarboxylic acids. Another type of builder, useful in granular laundry and built liquid laundry agents, includes various substantially water-insoluble materials which are capable of reducing the water hardness e.g. by ion exchange processes. In particular the complex sodium aluminosilicates, known as type A zeolites, are very useful for this purpose.

The formulations may also contain percompounds with a bleaching action, such as perborates, percarbonates, persulfates and organic peroxy acids. Formulations containing percompounds may also contain stabilizing agents, such as magnesium silicate, sodium ethylenediaminetetraacetate or sodium salts of phosphonic acids. In addition, bleach activators may be used to increase the efficiency of the inorganic persalts at lower washing temperatures. Particularly useful for this purpose are substituted carboxylic acid amides, e.g., tetraacetylethylenediamine, substituted carboxylic acids, e.g., isononyloxybenzenesulfonate and sodiumcyanamide.

Examples of suitable hydrotropic substances are alkali metal salts of benzene, toluene and xylene sulfonic acids; alkali metal salts of formic acid, citric and succinic acid, alkali metal chlorides, urea, mono-, di-, and triethanolamine. Examples of solubilizer alcohols are ethanol, isopropanol, mono- or polyethylene glycols, monoproylene glycol and etheralcohols.

Examples of foam control agents are high molecular weight fatty acid soaps, paraffinic hydrocarbons, and silicon containing defoamers. In particular hydrophobic silica particles are efficient foam control agents in these laundry detergent formulations.

Examples of known enzymes which are effective in laundry detergent agents are protease, amylase and lipase. Preference is given to the enzymes which have their optimum performance at the design conditions of the washing and cleaning agent.

A large number of fluorescent whiteners are described in the literature. For laundry washing formulations, the derivatives of diaminostilbene disulfonates and substituted distyrylbiphenyl are particularly suitable.

As antigreying agents, water soluble colloids of an organic nature may preferably be used. Examples are water soluble polyanionic polymers such as polymers and copolymers of acrylic and maleic acid, cellulose derivatives such as carboxymethyl cellulose and methyl- and hydroxy-ethylcellulose.

In addition to one or more of the aforementioned other surfactants and other detergent composition components, compositions according to the invention typically comprise one or more inert components. For instance, the balance of liquid detergent composition is typically an inert solvent or diluent, most commonly water. Powdered or granular detergent compositions typically contain quantities of inert filler or carrier materials.

The invention also provides a process for preparing alkyl aromatic hydrocarbons which comprises contacting olefins with an aromatic hydrocarbon under alkylating conditions effective to alkylate said aromatic hydrocarbon. One process for preparing alkyl aromatic hydrocarbons is described in U.S. Pat. No. 6,747,165, which is herein incorporated by reference in its entirety.

The preparation of alkyl aromatic hydrocarbons by contacting the product olefins with aromatic hydrocarbons may be performed under a large variety of alkylating conditions. Preferably, the said alkylation leads to monoalkylation, and only to a lesser degree to dialkylation or higher alkylation, if any.

The aromatic hydrocarbon applicable in the alkylation may be one or more of benzene; toluene; xylene, for example o-xylene or a mixture of xylenes; and naphthalene. Preferably, the aromatic hydrocarbon is benzene.

The molar ratio of the olefins to the aromatic hydrocarbons may be selected from a wide range. In order to favor monoalkylation, this molar ratio is suitably at least about 0.5, preferably at least about 1, in particular at least about 1.5 In practice this molar ratio is frequently less than about 1000, more frequently less than about 100.

The said alkylation may or may not be carried out in the presence of a liquid diluent. Suitable diluents are, for example, paraffin mixtures of a suitable boiling range, such as the paraffins which were not converted in the dehydrogenation and which were not removed from the dehydrogenation product. An excess of the aromatic hydrocarbon may act as a diluent.

The alkylation catalyst, which may be applied, may be selected for example from a large range of zeolitic alkylation catalysts. In order to favor monoalkylation, it is preferred that the zeolitic alkylation catalysts have pore size dimensions in the range of from about 4 to about 9 Å, more preferably from about 5 to about 8 Å and most preferably from about 5.5 to about 7 Å, on the understanding that when the pores have an elliptical shape, the larger pore size dimension is the dimension to be considered. The pore size dimensions of zeolites has been specified in W M Meier and D H Olson, “Atlas of Zeolite Structure Types”, 2^{nd} and revised edition (1987), published by the Structure Commission of the International zeolite Association. Suitable zeolitic alkylation catalysts are zeolites in acidic form selected from zeolite Y and zeolites ZSM-5 and ZSM-11. Preferably the zeolitic alkylation catalysts are zeolites in acidic form selected from mordenite, ZSM-4, ZSM-12, ZSM-20, offretite, gemelinite and cancrinite. Particularly preferred zeolitic alkylation catalysts are the zeolites which have an NES zeolite structure type, including isotypic framework structures such as NU-87 and gottardiute, as disclosed in U.S. Pat. No. 6,111,158. The zeolites which have an NES zeolite structure type give, advantageously, a high selectivity to 2-aryl-alkanes. Further examples of suitable zeolitic alkylation catalyst have been given in WO-99/05082, which is herein incorporated by reference.

Processes for treatment the zeolitic alkylation catalyst or of precursors thereof to prepare an active form of the zeolitic alkylation catalyst are given in WO-99/05082, which is herein incorporated by reference. Examples of such treatments are ion exchange reactions, dealumination, steaming, calcination in air, in hydrogen or in an inert gas, and activation. Specific information on how these catalysts may be used is given in U.S. Pat. No. 6,747,165, which is herein incorporated by reference in its entirety.

The preparation of alkyl aromatic hydrocarbons by contacting the olefins with the aromatic hydrocarbon may be performed under alkylating conditions involving reaction temperatures selected from a large range. The reaction temperature is suitably selected in the range of from about 30° C. to about 300° C., more suitably in the range of from about 100° C. to about 250° C.

Work-up of the alkylation reaction mixture may be accomplished by methods known in the art. For example, a solid catalyst may be removed from the reaction mixture by filtration or centrifugation. Unreacted hydrocarbons, for example olefins, any excess of intake aromatic hydrocarbons or paraffins, may be removed by distillation.

The general class of branched alkyl aromatic compounds which may be made in accordance with this invention may be characterized by the chemical formula R-A, wherein R represents a radical derived from the branched olefins according to this invention by the addition thereto of a hydrogen atom, which branched olefins may have a carbon number in the range of from about 7 to about 35, in particular from about 7 to about 18, more in particular from about 10 to about 18, most in particular from about 11 to about 14; and A represents an aromatic hydrocarbyl radical, in particular a phenyl radical.

The invention also provides a process for preparing alkylarylsulfonates comprising sulfonating the alkyl aromatic hydrocarbons described above. One process for preparing alkylarylsulfonates is described in U.S. Pat. No. 6,747,165, which is herein incorporated by reference in its entirety.

The alkyl aromatic compounds of this invention may be sulfonated by any method of sulfonation which is known in the art. Examples of such methods include sulfonation using sulfuric acid, chlorosulfonic acid, oleum or sulfur trioxide. Details of a preferred sulfonation method, which involves using an air/sulfur trioxide mixture, are known from U.S. Pat. No. 3,427,342, which is herein incorporated by reference.

Any convenient work-up method may be employed after the sulfonation. The sulfonation reaction mixture may be neutralized with a base to form the alkylarylsulfonate in the form of a salt. Suitable bases are the hydroxides of alkali metals and alkaline earth metals; and ammonium hydroxides, which provide the cation M of the salts as specified below.

The general class of alkylarylsulfonates which may be made in accordance with this invention can be characterized by the chemical formula (R-A′-SO₃)ₙM, wherein R represents a radical derived from the olefin products by the addition thereto of a hydrogen atom, which olefins may have a carbon number in the range of from about 7 to about 35, in particular from about 7 to about 18, more in particular from about 10 to about 18, most in particular from about 11 to about 14; A′ represents a divalent aromatic hydrocarbyl radical, in particular a phenylene radical; M is a cation selected from an alkali metal ion, an alkaline earth metal ion, an ammonium ion, and mixtures thereof; and n is a number depending on the valency of the cation(s) M, such that the total electrical charge is zero. The ammonium ion may be derived from an organic amine having 1, 2 or 3 organic groups attached to the nitrogen atom. Suitable ammonium ions are derived from monoethanol amine, diethanol amine and triethanol amine. It is preferred that the ammonium ion is of the formula NH₄⁺. In preferred embodiments M represents potassium or magnesium, as potassium ions can promote the water solubility of the alkylarylsulfonates and magnesium can promote their performance in soft water.

The alkylarylsulfonate surfactants which can be made in accordance with this invention may be used as surfactants in a wide variety of applications, including detergent formulations such as granular laundry detergent formulations, liquid laundry detergent formulations, liquid dishwashing detergent formulations; and in miscellaneous formulations such as general purpose cleaning agents, liquid soaps, shampoos and liquid scouring agents.

The alkylarylsulfonate surfactants find particular use in detergent formulations, specifically laundry detergent formulations. These formulations are generally comprised of a number of components besides the alkylarylsulfonate surfactants themselves: other surfactants of the ionic, nonionic, amphoteric or cationic type, builders, cobuilders, bleaching agents and their activators, foam controlling agents, enzymes, anti-greying agents, optical brighteners, and stabilizers. These detergent formulations may comprise many of the same components described above.

The liquid laundry detergent formulations may comprise the same components as the granular laundry detergent formulations, but they generally contain less of the inorganic builder component. Hydrotropes may be present in the liquid detergent formulations. General purpose cleaning agents may comprise other surfactants, builders, foam control agents, hydrotropes and solubilizer alcohols. These detergent formulations may comprise many of the same components described above.

### EXAMPLES

In these examples, higher boiling internal olefin products, which are mid-chain internal olefins, are made by allowing an internal olefin to self-metathesize over a non-isomerizing catalyst under non-equilibrium conditions where the lower boiling internal olefin product is flashed off as it is formed. The higher boiling internal olefin product (mid chain internal olefins) is separated from the paraffins by distillation, thereby producing a desired mid-chain olefin product with a higher carbon number (C₁₄) than the feed internal olefin and an olefin product with a lower carbon number (represented below by —C₄) than the feed internal olefin as shown in equation 1 below,

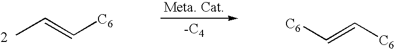

Table 1 describes the conditions and results for a number of experiments which are carried out using an 11 percent by weight Re/Al₂O₃ catalyst as the metathesis catalyst.

Examples 1, 3 and 5 utilize NEODENE® 1112 olefin (an internal olefin containing a mixture of C₁₁ and C₁₂ internal olefins) as the internal olefin in the experiment. Example 1 shows the production of C₁₃₋₂₀ mid chain olefins (referred to as “MCO” in Table 1) from neat NEODENE® 1112 internal olefin and from the same internal olefin diluted with paraffin to simulate the product of paraffin dehydrogenation by the Pacol process. It can be seen that the presence of paraffin in Example 3 has no significant effect on the conversion and selectivity to the mid-chain olefin. The parenthetical notations for Examples 3 and 5 in the LHSV column provide the LHSV for the olefin only in the olefin/paraffin mixtures.

Examples 2 and 4 are carried out using DIMERSOL® dodecene (referred to as “DIM.” in the IO feed column) as the feed internal olefin. This material had previously been reported to be relatively unreactive in metathesis but in this reaction good conversion is achieved and good selectivity to olefins of a higher carbon number than dodecene is achieved when it is freshly distilled and metathesized over the catalyst previously described. Heavier, higher boiling olefins, containing four branches per chain according to NMR analysis, are recovered.

TABLE 1
Mid-Chain Olefin (MCO) Preparation
Catalyst, 11% Re/Al₂O₃

| | | | | | | | | Selectivity, % wt | | |
|---|---|---|---|---|---|---|---|---|---|---|
| Exam- | LR- | | Temp., | Vac., | | | Conv., | | C-13/20 | |
| ple | 22964 | IO Feed | ° C. | mmHg | | LHSV | % wt | ≦C-10 IO | MCO | Comments |
| 1 | 120 | C-11/12^{d} | 44 | 2 | (.3 kPa) | 1.0 | 79.1 | 29^{a} | 71^{b} | |
| 2 | 143 | Dim. Deoct. Bot^{f} | 45 | 3 | (.4 kPa) | 0.5 | Low | | | 2492 ppm TBT in feed^{c} |
| 3 | 145 | 15% wt C-11/12^{d} | 45 | 10 | (1.3 kPa) | 3.3 | 80 | 27.5^{a} | 72.5^{b} | 1243 ppm TBT in feed |
| | | in cane | | | | (0.5 on | | | | |
| | | | | | | olefin) | | | | |
| 4 | 153 | Dim. C-12^{c} | 45 | 5–8 | (.67–1.07 kPa) | 0.5 | 68.2 | 35.2^{a} | 64.8^{b} | 2500 ppm TBT in feed |
| 5 | 170 | 15% wt C-11/12^{d} | 45 | 9–10 | (1.2–1.3 kPa) | 6.7 | 77.4 | 29^{a} | 71^{b} | 1464 ppm TBT in feed |
| | | in cane | | | | (1.0 on | | | | |
| | | | | | | olefin) | | | | |

^{a}Lower boiling than feed component
^{b}Higher boiling than feed component
^{c}TBT, tetrabutyl tin
^{d}NEODENE ® 1112 olefin
^{e}DIMERSOL ® Dodecene
^{f}DIMERSOL ® Dodecene deoctanizer bottoms - the bottoms from the deoctanizer column that separates the higher bowling greater than C8 branched olafins as the bottoms

We claim:

1. A process comprising:
(a) providing a mixture comprising feed olefins and non-olefinic organic compounds, and
(b) converting the feed olefins in the mixture to converted olefins to increase the difference in boiling point of the olefins from the non-olefinic organic compounds.

2. The process of claim 1 wherein the feed olefins are converted into converted olefins for which the difference in boiling point from the boiling point of the non-olefinic organic compounds is larger than the difference in boiling point of the feed olefins from the boiling point of the non-olefinic organic compounds.

3. The process of claim 2 wherein the difference in the boiling points of the feed olefins and the non-olefinic organic compounds is about 10° C. or less.

4. The process of claim 2 wherein the difference in the boiling points of the converted olefins and the non-olefinic organic compounds is more than about 5° C.

5. The process of claim 2 wherein the difference in the boiling points of the converted olefins and the non-olefinic organic compounds is more than about 10° C.

6. The process of claim 2 wherein an additional step c) is performed which comprises separating the converted olefins from the mixture produced in step b).

7. The process of claim 6 wherein the converted olefins comprise lower boiling olefin products and higher boiling olefin products and step c) comprises
c) (i) separating the lower boiling olefin products from the mixture produced in step b), and
c) (ii) separating the higher boiling olefin products from the remaining material.

8. The process of claim 7 wherein steps b) and c) (i) are carried out together at the same time.

9. The process of claim 2 wherein at least a portion of the converted olefins are separated from the reaction mixture of step b) as they are formed.

10. The process of claim 6 wherein steps b) and c) are carried out in the same reaction vessel.

11. The process of claim 6 wherein all of the steps are carried out in the same reaction vessel.

12. The process of claim 2 wherein the olefins are comprised of internal olefins.

13. The process of claim 12 wherein the internal olefins are comprised of mid-chain internal olefins.

14. The process of claim 2 wherein the mixture of step a) is a single carbon number cut.

15. A process comprising:
(a) providing a mixture comprising feed olefins and paraffins, and
(b) metathesizing the feed olefins in the mixture to converted olefins to increase the difference in boiling point of the olefins from the paraffins.

16. The process of claim 15 wherein the feed olefins are metathesized into converted olefins which comprise lower boiling olefin products and higher boiling olefin products for which the difference in boiling point from the boiling point of the paraffins is larger than the difference in boiling point of the feed olefins from the boiling point of the paraffins.

17. The process of claim 16 wherein the difference in boiling points of the feed olefins and the paraffins is about 10° C. or less.

18. The process of claim 16 wherein the difference in the boiling points of the converted olefins and the paraffins is more than about 5° C.

19. The process of claim 16 wherein the difference in the boiling points of the converted olefins and the paraffins is more than about 10° C.

20. The process of claim 16 wherein an additional step c) is performed which comprises separating the converted olefins from the mixture produced in step b).

21. The process of claim 20 wherein the converted olefins comprise lower boiling olefin products and higher boiling olefin products and step c) comprises
c) (i) separating the lower boiling olefin products from the mixture produced in step b), and
c) (ii) separating the higher boiling olefin products from the remaining material.

22. The process of claim 21 wherein steps b) and c) (i) are carried out together at the same time.

23. The process of claim 16 wherein the lower boiling olefin product is removed from the reaction mixture of step b) as it is formed.

24. The process of claim 16 wherein steps b) and c) are carried out in the same reaction vessel.

25. The process of claim 16 wherein all of the steps are carried out in the same reaction vessel.

26. The process of claim 16 wherein the olefins are comprised of internal olefins.

27. The process of claim 26 wherein the internal olefins are comprised of mid-chain internal olefins.

28. The process of claim 16 wherein the metathesis is carried out at a temperature from about −10° C. to about 300° C.

29. The process of claim 16 wherein the metathesis catalyst is a tungsten-based catalyst is used and the temperature is from about 200 to about 300° C.

30. The process of claim 16 wherein the metathesis catalyst is a molybdenum-based catalyst is used and the metathesis is carried out at a temperature from about 100 to about 150° C.

31. The process of claim 16 wherein the metathesis catalyst is a rhenium-based catalyst is used and the metathesis is carried out at a temperature from about 30 to about 60° C.

32. The process of claim 16 wherein the mixture of step a) is a single carbon number cut.

33. The process of claim 16 wherein a metathesis catalyst is used and the metathesis catalyst is comprised of one or more—metals selected from the group consisting of Mo, W, Re, and Ru.

34. The process of claim 23 wherein the metathesis is carried out under non-equilibrium conditions.

35. The process of claim 34 wherein a metathesis catalyst is used and the metathesis catalyst is a non-isomerizing metathesis catalyst.

36. The process of claim 35 wherein the non-isomerizing metathesis catalyst is selected from the group consisting of heterogeneous catalysts in which rhenium, molybdenum or tungsten is deposited on a support of silica, alumina, or alumina phosphate and homogeneous catalysts based on ruthenium.

37. The process of claim 36 wherein the non-isomerizing metathesis catalyst is comprised of rhenium deposited on alumina.

38. The process of claim 35 wherein the non-isomerizing metathesis catalyst is a Grubbs catalyst.

39. The process of claim 35 wherein the metathesis catalyst is comprised of one or more metals selected from the group consisting of Mo, W, Re, and Ru.

40. The process of claim 16 wherein a metathesis catalyst is used and the metathesis catalyst is a non-isomerizing metathesis catalyst.

41. The process of claim 40 wherein the non-isomerizing metathesis catalyst is selected from the group consisting of heterogeneous catalysts in which rhenium, molybdenum or tungsten is deposited on a support of silica, alumina, or alumina phosphate and homogeneous catalysts based on ruthenium.

42. The process of claim 41 wherein the non-isomerizing metathesis catalyst is comprised of rhenium deposited on alumina.

43. The process of claim 40 wherein the non-isomerizing metathesis catalyst is a Grubbs catalyst.

44. The process of claim 16 wherein the feed olefins and paraffins provided in step a) are of the same carbon number.

45. A process for producing derivatives of olefins which comprises:
(a) providing a mixture comprising feed olefins and non-olefinic organic compounds,
(b) converting the feed olefins in the mixture to converted olefins to increase the difference in boiling point of the olefins from the non-olefinic organic compounds,
(c) recovering the olefin products, and
(d) either:
(i) hydroformylating the olefin products to produce alcohols; or
(ii) hydroformylating the olefin products to produce alcohols, and adding an alkylene oxide to the alcohols in the presence of an alkoxylation catalyst to produce alcohol alkoxylates; or
(iii) hydroformylating the olefin products to produce alcohols, adding an alkylene oxide to the alcohols in the presence of an alkoxylation catalyst, and sulfonating the alcohol alkoxylates; or
(iv) hydroformylating the olefin products to produce alcohols, and sulfonating the alcohols; or
(v) contacting the olefin products with aromatic hydrocarbons under alkylating conditions effective to alkylate the aromatic hydrocarbons to produce alkyl aromatic hydrocarbons; or
(vi) contacting the olefin products with aromatic hydrocarbons under alkylating conditions effective to alkylate the aromatic hydrocarbons to produce alkyl aromatic hydrocarbons, and sulfonating the alkyl aromatic hydrocarbons to produce alkylarylsulfonates; or
(vii) sulfonating the olefin products to produce sulfated olefins; or
(viii) hydroformylating the olefin products to produce alcohols, adding an alkylene oxide to the alcohols in the presence of an alkoxylation catalyst to produce alcohol alkoxylates, and combining the alcohol alkoxylates with conventional detergents additives; or
(ix) hydroformylating the olefin products to produce alcohols, adding an alkylene oxide to the alcohols in the presence of an alkoxylation catalyst to produce alcohol alkoxylates, sulfonating the alcohol alkoxylates, and combining the sulfonated alcohol alkoxylates with conventional detergents additives; or
(x) hydroformylating the olefin products to produce alcohols, sulfonating the alcohols, and combining the sulfonated alcohols with conventional detergents additives; or
(xi) contacting the olefin products with aromatic hydrocarbons under alkylating conditions effective to alkylate the aromatic hydrocarbons to produce alkyl aromatic hydrocarbons, sulfonating the alkyl aromatic hydrocarbons to produce alkylarylsulfonates, and combining the alkylarylsulfonates with conventional detergents additives.
